(19) United States
(12) Patent Application Publication
(10) Pub. No.: US 2018/0193105 A1
(43) Pub. Date: Jul. 12, 2018

(54) CLEAN MEDICAL DEVICE CASE

(71) Applicant: MilSpec Communications, Inc., Beaufort, SC (US)

(72) Inventor: Howard L. Bayne, III, Beaufort, SC (US)

(21) Appl. No.: 15/861,923

(22) Filed: Jan. 4, 2018

Related U.S. Application Data
(60) Provisional application No. 62/443,225, filed on Jan. 6, 2017.

(51) Int. Cl. A61B 50/31; B65D 81/18; B65D 43/12; B65D 55/10; B65D 25/54; B65D 25/04

(52) U.S. Cl. CPC A61B 50/31; B65D 81/18; B65D 43/12; A61B 2050/311; B65D 25/54; B65D 25/04; B65D 55/10

(57) ABSTRACT

A clean medical device case is described herein, which includes embodiments for use both inside and outside traditional hospital settings. The clean medical device case includes an insert, lid, and locking mechanism. The insert is formed to receive a medical device and the case keeps the medical device sterile for storage and safe for transit. Wiping and soaking methods for cleaning the clean medical device case are also disclosed.

### CROSS REFERENCE TO RELATED APPLICATIONS

This application claims the benefit of U.S. Provisional Application No. 62/443,225 filed Jan. 6, 2017, which is incorporated by reference in its entirety.

### TECHNICAL FIELD

The disclosure relates to the technical field of medical devices, and in particular, to a clean medical device case.

### BACKGROUND

Medical devices used by physicians to view the interior of a patient's body include various instruments such as arthoscopes, bronchoscopes, colonoscopes, colposcopes, cystocscopes, esophagoscopes, gastroscopes, laprascopes, laryngoscopes, neuroendoscopes, proctoscopes, sigmoidoscopes, and thoracoscopes. These medical scope devices may be used in operating rooms and similar medical environments both inside a traditional hospital setting and outside a traditional hospital setting, such as on a battlefield.

Medical scope devices (and similar devices) are required to be cleaned or sterilized between usage, and stored in a clean environment before use. These devices may also be highly expensive and prone to damage during shipping, usage, and cleaning. Notably, known devices and methods for protecting the devices are unable to provide a clean environment to the medical device. These known devices and methods are likewise unable to provide for safe and secure protection of the device during transit and use.

Today's laryngoscopes can cost over of $5,000 a piece. It is estimated that approximately 3-4% of these devices are damaged in the operating room every year. The shape and many components that comprise a medical device may also prevent the medical device from assuming a stable position on an operating table. Often, the medical device may be knocked to the floor from the operating table. Medical devices such as laryngoscopes may be difficult to repair, due at least in part to the unique shape of the device and the many components associated with it (i.e., battery, computer, monitor, etc.).

Furthermore, the medical devices used in the operating room must remain clean after cleaning in order to minimize the patient's exposure to dangerous infections. To do this, medical devices are currently enclosed within a sealed plastic bag after sterilization and then stored within the sealed plastic bag in the operating room. When the medical device is required for use, the medical device may be removed from the sealed plastic bag and placed on the operating table. However, the sealed plastic bag provides little to no protection to the enclosed medical device against damage due to transportation, storage and handling by hospital personnel. Furthermore, the plastic bag may not remain sterile if damaged. For example, these bags are easily pierced thereby forfeiting any sterile protection. For that reason, storing medical scope devices in plastic bags is discouraged by infection control specialists.

Accordingly, there is a need to provide a protective environment capable of transporting, storing, and/or using a medical device. Additionally, there is a need to provide a sterile environment for the medical device. Furthermore, as the medical device may need to be used in various medical environments including nontraditional battlefield locations, there is a need for the protective equipment to be both sterile and ambulatory.

### SUMMARY

The following describes preferred embodiments of a clean medical device case that is designed and shaped to protect a medical device from damage, for example during transportation and use. Contemporaneously, the clean medical device case provides an environment that allows the medical device to be cleaned and sterilized, and remain sterilized while stored. These and other benefits will be apparent based on the following description.

In one embodiment, a clean medical device case includes an outer frame, an insert, and a lid. The insert may include a lip about the perimeter of the insert, and a body region that is configured to hold a medical device. The outer frame may have a first end that is spaced apart from a second end. A first groove may be located along an inner surface of the outer frame dividing the outer frame into an upper portion and a lower portion. The first groove may be configured to engage with the lip of the insert. A second groove may be located along an upper portion of the inner surface of the outer frame proximate the first end of the outer frame. The lid may be configured to releasably attach to the outer frame by engaging with the second groove. In one embodiment, the inner surface of the upper portion of the outer frame, the body region of the insert, and an interior surface of the lid may form a sterile environment.

In one embodiment, a clean medical device case includes an upper section hingedly connected to a lower section. The upper and lower sections may form a seal when the upper and lower sections are pivoted towards each other. An insert may be embedded within the lower section. The insert may be configured to hold a medical device. The inner surface of the upper section, the insert, and an upper portion of the lower section may form a sterile environment.

In one embodiment, a clean medical device case is manufactured by forming an insert through injection molding or a similar process. The insert comprises cavities sized to snugly hold a medical device. Four panels of an outer frame are also formed having an inside groove or channel sized to accept a lip of the insert. Adhesive is then optionally applied to the insert lip/panel groove interface (either on the insert or within the groove), and the panels are joined to the insert so that the insert's lip fits into the panel groove. The panels may then be clamped in place and welded together at their ends. The top circumference of the insert may additionally be adhered or caulked to the insides of the panels for additional protection from contamination. Optionally, a base panel may be joined to the bottom of the case and welded or adhered into place. One or more of the side panels further comprise a locking mechanism and groove or other means on their top portion configured to both accept a lid and lock it into a closed position.

In one embodiment, the clean medical device case may be manufactured using injection molding. Injection molding may allow for greater uniformity and consistency with standardized protocols. In one embodiment the outer frame and other components of the clean medical device may be made of thermoplastic acrylic-polyvinyl chloride materials such as those sold under the trademark Kydex®, or other like materials. In one embodiment, the use of Kydex® may eliminate the need for plastic welding. In such an embodiment, the components may be assembled using an adhesive or glue.

In one embodiment, a clean medical device case is manufactured by creating an insert configured to snugly hold a medical device, engaging and adhering the insert to a first groove of an outer frame comprising one or more panels and a first portion of a locking mechanism, welding the insert and outer frame such that the insert is embedded within the outer frame, creating a lid having a second portion of the locking mechanism, engaging the lid with the second groove of the outer frame, wherein the lid is held in place with respect to the outer frame when the first portion of the locking mechanism is engaged with the second portion of the locking mechanism.

In one embodiment, a medical device case is sterilized by removing the contents of the medical device case, filling a receptacle with a cleaning agent, immersing the medical device case in the cleaning agent, soaking the medical device case in the cleaning agent for a first time, agitating the medical device case within the cleaning agent, filling a second receptacle with a rinse solution, immersing the medical device case in the rinse solution such that the cleaning agent is removed from the medical device case, and drying the medical device case.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is an illustration of an embodiment of the clean medical device case in an unengaged state from a top perspective view.

FIG. 2 is an illustration of the embodiment of the clean medical device case depicted in FIG. 1 in an engaged state from a top perspective view.

FIG. 3 is an illustration of selected components of the clean medical device case depicted in FIGS. 1 and 2 from a top perspective view.

FIG. 4 is an illustration of selected components of the clean medical device case depicted in FIGS. 1-3 from a top perspective view.

FIG. 5 is an illustration of the embodiment of the clean medical device case depicted in FIGS. 1-4 from a top view.

FIG. 6 is an illustration of twelve friction feet, one or more of which may be used with an embodiment of the clean medical device case.

FIG. 7A illustrates an insert that may be used with an embodiment of the clean medical device case from a top view.

FIG. 7B illustrates the insert depicted in FIG. 7A from a top perspective view.

FIG. 7C illustrates the insert depicted in FIG. 7A-7B from a side view.

FIG. 7D illustrates the insert depicted in FIG. 7A-7C from a second side (lengthwise) perspective view.

FIG. 7E illustrates the insert depicted in FIG. 7A-7D from a bottom view.

FIG. 8A illustrates a second embodiment of the clean medical device case in a closed configuration from a top perspective view.

FIG. 8B illustrates the second embodiment of the clean medical device case depicted in FIG. 8A in a closed configuration from a bottom perspective view.

FIG. 8C illustrates the second embodiment of the clean medical device case depicted in FIGS. 8A and 8B in a closed configuration from a second bottom perspective view.

FIG. 8D illustrates the second embodiment of the clean medical device case depicted in FIGS. 8A-8C in a closed configuration from a second top perspective view.

FIG. 8E illustrates a second embodiment of the clean medical device case depicted in FIGS. 8A-8D in an open configuration from a top perspective view.

FIG. 8F illustrates a second embodiment of the clean medical device case depicted in FIGS. 8A-8E in an open configuration from a second top perspective view.

FIG. 8G illustrates a second embodiment of the clean medical device case depicted in FIGS. 8A-8F in an open configuration from a top view.

FIG. 8H illustrates a second embodiment of the clean medical device case depicted in FIGS. 8A-8G in an open configuration from a third top perspective view.

FIG. 8I illustrates an insert configured to be used with the second embodiment of the clean medical device case depicted in FIGS. 8A-8H from a top perspective view.

FIG. 8J illustrates an outer frame configured to be used with the second embodiment of the clean medical device case depicted in FIGS. 8A-8I from a top perspective view.

FIG. 9 illustrates an embodiment of the clean medical device case.

FIG. 10 illustrates another embodiment of the clean medical device case.

FIG. 11 illustrates a medical device as provided in its original non-sterile packaging.

FIG. 12 illustrates a medical device placed within an insert of a clean medical device case.

FIG. 13 illustrates a medical device placed within an insert of a clean medical device case.

FIG. 14 illustrates an insert of a clean medical device case.

FIG. 15 illustrates specifications for an insert of a clean medical device case.

FIG. 16 illustrates specifications for an outer frame of a clean medical device case.

FIG. 17 illustrates specifications for a lid and outer frame of a clean medical device case.

FIG. 18 illustrates specifications for a lid of a clean medical device case.

FIG. 19 illustrates specifications for a locking mechanism used in connection with a clean medical device case.

### DETAILED DESCRIPTION

A clean or sterile medical device case is described herein. The clean or sterile medical device case is able to provide a protective environment while transporting, storing, and/or using a medical device. Notably, the interior of the clean or sterile medical device case is sterile. Additionally, the clean or sterile medical device case may be portable. As used herein the word “sterile” may refer to an area that is free from bacteria or other living microorganisms.

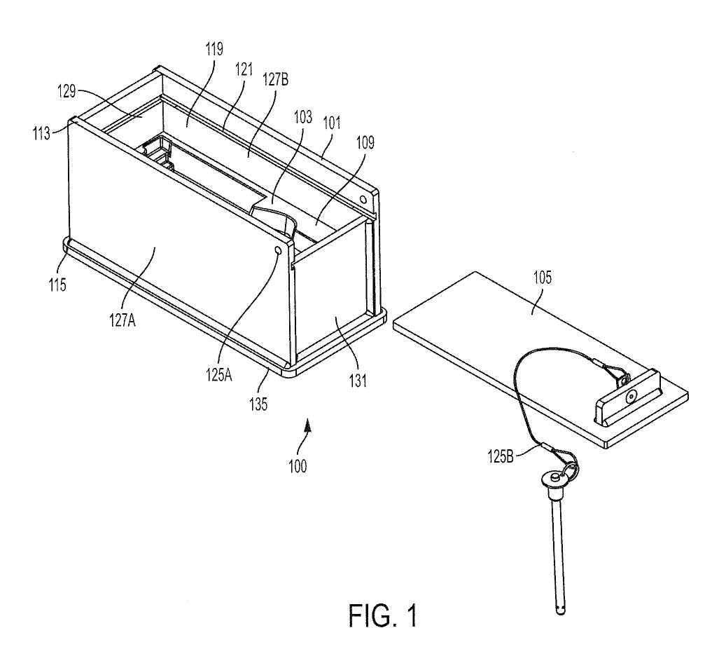

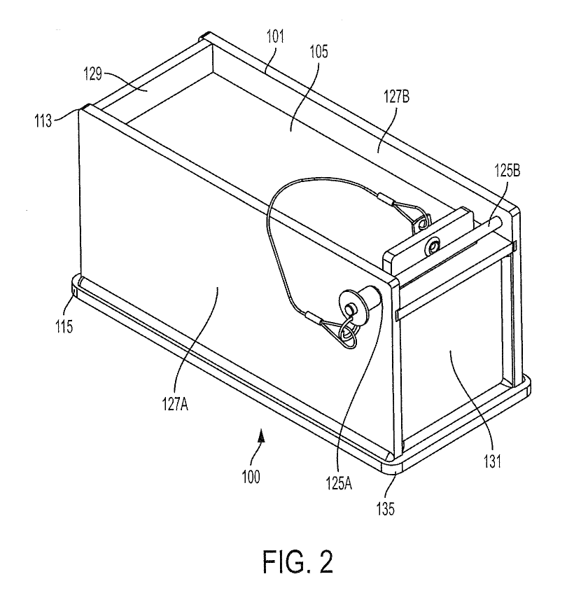

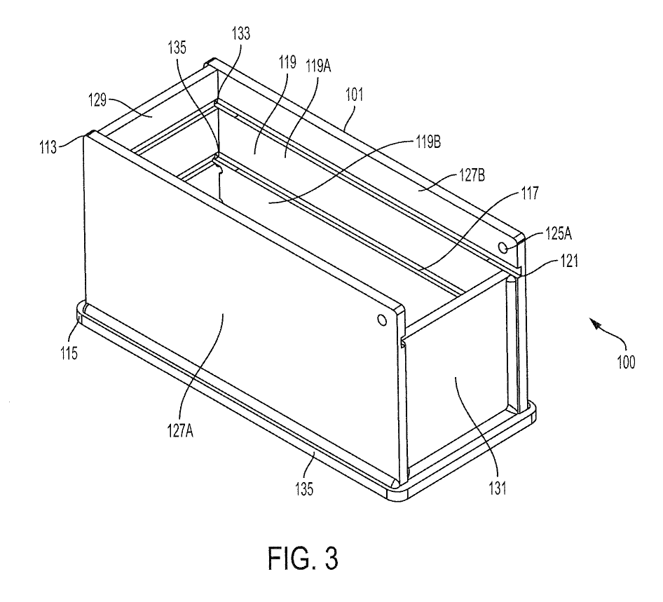

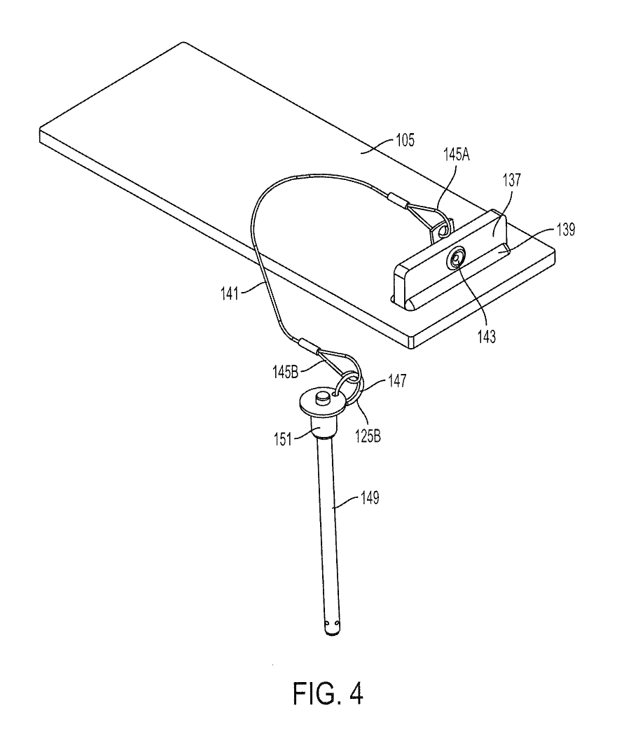

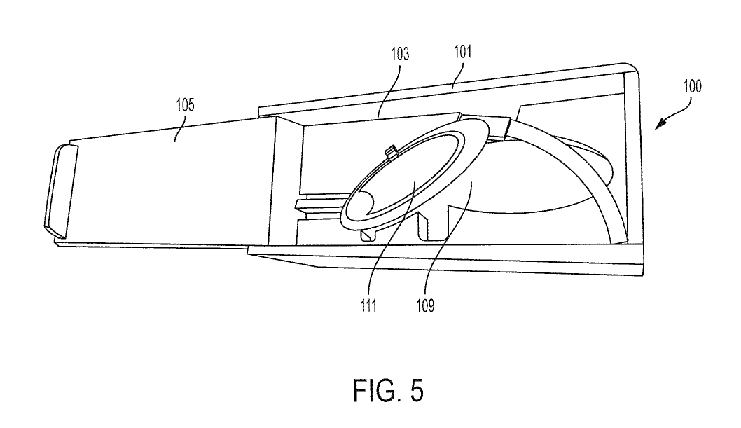

An embodiment of the clean medical device case is illustrated in FIGS. 1-4. More particularly, FIG. 1 illustrates the components of the clean medical device case 100: the outer frame 101 with insert 103 and the lid 105 in an unengaged state. FIG. 2 illustrates the clean medical device case 100 with the outer frame 101 and insert 103 engaged with the lid 105. FIG. 3 illustrates the outer frame 101 without the insert 103 and lid 105. FIG. 4 illustrates the lid 105. FIG. 5 illustrates the clean medical device case 100 from a top view.

The components illustrated in FIGS. 1-5 are discussed herein. The depicted clean medical device case 100 includes an outer frame 101, an insert 103, and a lid 105. The insert 103 may include a lip (not shown) about the perimeter of the insert 103 and a body region that is configured to hold a medical device 111 (shown in FIG. 5). The outer frame 101 may have a first end 113 that is vertically spaced apart from a second end 115. A first groove 117 (shown in FIG. 3) may be located along an inner surface 119 of the outer frame 101 dividing the outer frame 101 into an upper portion 119A and a lower portion 119B. The first groove 117 may be configured to engage with the lip (not shown) of the insert 103.

A second groove 121 may be located along an upper portion 119A of the inner surface 119 of the outer frame 101 proximate the first end 113 of the outer frame 101. The lid 105 may be configured to releasably attach to the outer frame 101 by engaging with the second groove 121. The lid 105 may be engaged to the outer frame 101 by mating a locking component 125B (see FIG. 4) of the lid with a receiving component 125A (see FIG. 3) on the outer frame 101. The locking component 125B of the lid may be any suitable locking mechanism such as, for example, a pin, bolt, latch, and the like. The lid 105 may be formed with a suitable transparent material, such as acrylic glass, to allow a user to see the medical device contain in the clean medical device case 100.

In one embodiment, the lid may be separable and independent from the outer frame. Accordingly, the lid may not be connected to the outer frame via a cord or lanyard. Instead, the lid may be attached to the outer frame and locked in place using a stainless steel spring loaded barrel lock, or other fastening or locking means generally known in the industry.

In one embodiment, components of the clean medical device case 100 may form one or more sterile environments. A first sterile environment may be formed in the volume enclosed by the top surface of the insert 103, interior surface of the lid 105 (i.e., the surface configured to face the insert when the lid is engaged with the outer frame 101), and the surface formed by the upper portion 119A of the outer frame 101. A second sterile environment may be formed in the volume enclosed by the bottom surface of the insert 103, the surface formed by the lower portion 119B of the outer frame 101, and a fifth panel 135.

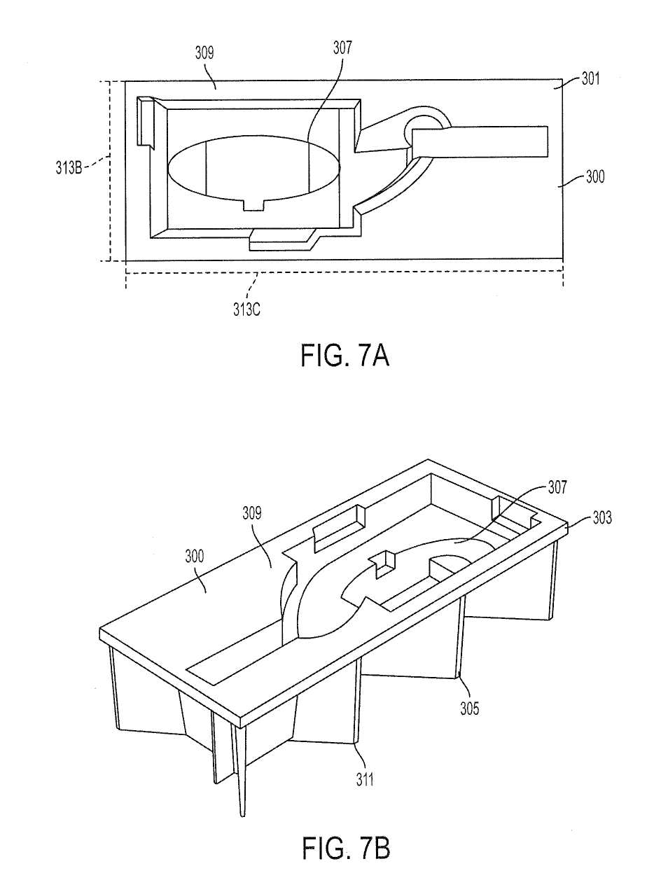

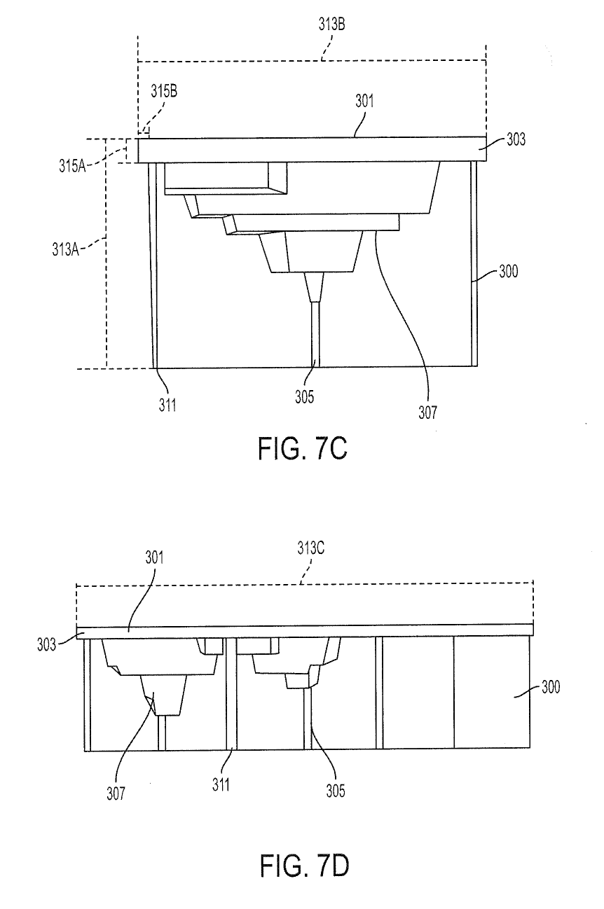

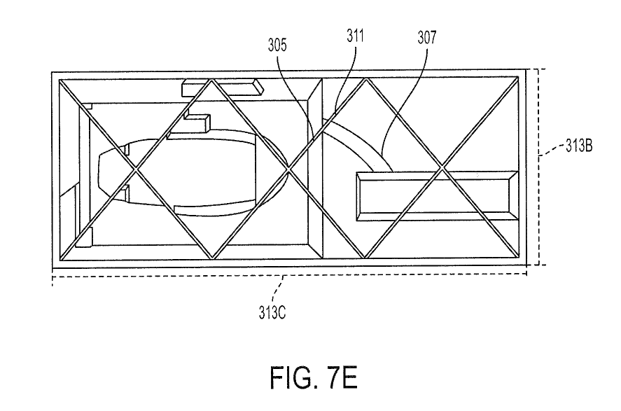

The outer frame 101 may substantially surround the insert 103 (discussed in further detail with regards to FIGS. 7A-7E). The outer frame may be constructed from high density polyethelyne (HDPE), ultra high molecular weight (UHMW) plastic, or the like. The outer frame may include one or more panels that are manufactured by 3D printing or other means. In one embodiment the outer frame is constructed from HDPE having a thickness of approximately 6.35 mm. It is envisioned that any suitable materials and thickness may be used.

In one embodiment, the outer frame 101 may form a substantially rectangular box with an open top surface. The outer frame may be constructed from five separate panels. As will be discussed below, the panels of the outer frame 101 may be configured to align with and form a layer around the sides and bottom of the insert 103. The outer frame 101 may include two long side panels 127A, 127B that extend parallel to each other and may be separated by the width of the insert 103. A first end of each of the two long side panels 127A, 127B may terminate at a rear short side panel 129 located at a first end of the clean medical device case. A second end of the two long side panels 127A, 127B may terminate at a front short side panel 131 that is located at a second end of the clean medical device case. One of the two ends of the clean medical device case may be proximate to a locking mechanism 125A.

Each of the two long side panels 127A, 127B and the rear short side panel 129 may be constructed to have a height that is greater than the height of the insert. The two long side panels 127A, 127B may be constructed so that each has a length longer than that of the insert, for example by twice the thickness of the outer frame 101 material (discussed below). The rear short side panel 129 may be constructed to have a length that is corresponding to the width of the insert. The front short side panel 131 may be constructed to have a length that corresponds to the width of the insert and a height that is less than the height of the other lateral panels (the two long side panels 127A, 127B, and the rear short side panel 129). The front short side panel 131 may play a role in securing a lid to the outer frame (discussed below).

In one embodiment, the outer frame may also include a fifth panel 135 having dimensions such that the fifth panel has an area exceeding the area of the insert 103. In particular, in one embodiment, the fifth panel 135 may have a length corresponding to that of the insert 103 plus, for example, the thickness of the front short side panel 131 and the thickness of the rear short side panel 129. Additionally, the fifth panel 135 may have a width corresponding to that of the insert 103, plus, for example, the thickness of the two long side panels 127A, 127B. The fifth panel 135 may form the base of the outer frame. In one embodiment, the fifth panel 135 may be oversized to facilitate the sealing of the insert 103 within the outer frame 101, and the welding of the panels that make up the outer frame 101 (127A, 127B, 129, 131) with respect to each other. In one embodiment, where the outer frame 101 is constructed from HDPE, the outer frame may have a weight of about 300-600 grams.

The four panels (the two long side panels 127A, 127B, the rear short side panel 129, and the front short side panel 131) that form the lateral sides of the outer frame 101 may each include a first groove that recesses from the panel and is positioned to be on the inner surface of the outer frame. The groove on each respective panel may be positioned so that it aligns with the lip of the insert (discussed below) and with the grooves on each of the other panels 135. The groove may be configured to receive a portion of the lip of the insert. The groove may divide the inner surface 119 of the outer frame 101 into an upper portion 119A and a lower portion 119B. Notably, the upper portion 119A of the inner surface 119 of the outer frame 101 may be used to provide a sterile environment to a medical device. The lower portion 119B of the inner surface 119 of the outer frame 101 may also be used to provide a second sterile environment.

In one embodiment, the outer frame may have a second groove 121 that spans the inner surface 119 of the outer frame 101. The second groove 121 may recess from all three of the lateral panels (the two long side panels 127A, 127B, and the rear short side panel 129). The second groove 121 on each respective panel may be positioned so that they align with each other 133. The second groove 121 may be located along the upper portion 119A of the inner surface 119 of the outer frame 101 at a position that is at the height of the front short side panel 131. The second groove 121 may be configured to hold a lid 105. In one embodiment the second groove 121 configured to engage with a lid 105 may be placed such that there is a space of approximately 0.8 mm between the peak of the medical device held by the insert 103 and the lid 105. In one embodiment, the lid 105 may engage with the second groove 121 in a substantially sliding motion until the end of the lid 105 not having the locking mechanism is in contact with and seated in the rear short side panel 129.

In one embodiment, the lid 105 is also constructed from HDPE having a thickness of approximately 6.35 mm, although it is envisioned that any suitable materials and thickness may be used. The lid 105 may include a locking component 125B. The locking component 125B may be configured to mate with a receiving component 125A located on the outer frame 101. In the embodiment depicted in FIGS. 1-5, the locking component 125B includes a pull 137 that is welded to the lid about the surface 139. A rivet 143 located on the pull may hold a stainless steel component with an aperture that is configured for attachment to a lanyard 141. The lanyard 141 may have a first end terminating in a clasp 145A configured to mate with the aperture attached to the rivet 143. A second end 145B of the lanyard 141 may include a clasp configured to attach to a hook 147 attached to a pin 149. The pin 149 may have a first end including a cap 151 that holds the hook 147 and is used to maintain contact between the pin 149 and the lid 105. A second end of the pin 149 may be configured to mate with the receiving component 125A on the outer frame 101. In one embodiment, the receiving component 125A of the outer frame 101 includes two apertures, located on the opposing long side panels 127A 127B. The pin 149 may be inserted through the two apertures such that the lid 105 cannot be disengaged from the outer frame 101.

Although an embodiment of the locking mechanism including a lanyard, pin, and corresponding apertures on an outer frame 101 are depicted, it is contemplated that any suitable alternatives may be used. For example, in one embodiment the locking mechanism may include a safety-pin-like configuration. In another embodiment, the lanyard may be optional. In yet another embodiment, excess plastic material may be located in an interior corner of the outer frame 101 immediately below the second groove 121. The excess plastic material may include an aperture that is configured to line up with a corresponding aperture located along the lid 105. A pin may be inserted in a generally vertical motion through the aperture on the lid into the aperture in the excess plastic material. In the illustrated embodiment, the components of the locking component are composed of stainless steel. However, alternative materials and locking mechanisms may be used, such as, for example, acrylic glass and bolts, latches, and the like. It may be beneficial to use materials that can withstand the cleaning protocol described below.

In one embodiment, where the lid 105 is constructed from HDPE and the locking mechanism is constructed from stainless steel, the lid 105 assembly may have a weight of about 100-200 grams for the HDPE portions of the lid 105.

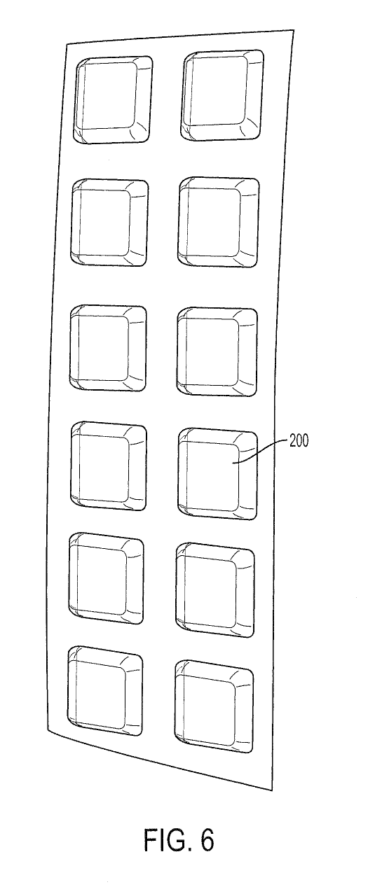

FIG. 6 illustrates an embodiment of friction feet that may be applied to the base of the outer frame 101. In the pictured embodiment, each of the twelve depicted friction feet elements 200 may be attached to the fifth panel 135 that forms the base of the outer frame 101. In one embodiment, four of the friction feet may be attached to the four corners of the fifth panel 135, which forms the base of the outer frame 101. The depicted friction feet elements 200 may use adhesive to attach to the base of the outer frame 101. The friction feet create additional friction between the clean medical device case 100 and the surface on which the clean medical device case 100 is placed (e.g., operating table, platform, etc.). The friction feet elements 200 aid in preventing the clean medical device case 100 from being dislodged from its intended position and add stability to the clean medical device case 100. For example, the friction feet may aid in preventing a medical device contained within the clean medical device case 100 from sliding off a table and being knocked to the ground or other hard surface. In one embodiment, each friction foot is approximately ⅜″×⅜″ in size. Alternative sizes and material may be used such as, for example, rubber or textured material. The friction feet may also take the form of a securement mechanism in union with a table such as, for example, fabric hook and loop fasteners, dowels, snaps, and the like. Also, a thin slab of silicone attached to the bottom of fifth panel 135 may provide ample friction proofing of clean medical device case 100 to, for example, an operating room table. This thin slab of silicone may be in addition to, or replace friction feet.

FIGS. 7A-7E illustrate various views of an insert 300 capable of being used with the clean medical device case 100. In particular, FIG. 7A illustrates a top view of an insert that may be used with an embodiment of the clean medical device. The insert depicted in FIGS. 7A-7E may be configured to hold a medical device (see, for example, FIG. 5). In one embodiment, the insert 300 may have a generally rectangular shape. A top surface 301 of the insert 300 may include a body region 309 having one or more cavities 307 each having a shape that conforms to one or more components of a medical device. For example, the one or more cavities 307 may be configured to hold one or more batteries, computer devices, blades, extensions, and monitors of the medical device. The one or more cavities 307 may recess from the top surface 301 towards the interior of the insert 300.

FIG. 7B illustrates a top perspective view of the insert depicted in FIG. 7A. The insert 300 may include one or more support members 305 that extend perpendicularly from the top surface 301 of the insert 300 in a generally downwards direction. The support members are preferably used to create voids in-between the member to lower the weight of the clean medical device case 100. However, the entire area may be one solid piece, or the insert 300 may be attached to the inner surface 119 of the outer frame 101.

The one or more support members 305 that extend generally perpendicular from the top surface 301 of the insert 300 are illustrated in the first side view depicted in FIG. 7C and the second side view depicted in FIG. 7D.

The top surface 301 of the insert 300 may also include a lip 303 that extends outwards along the perimeter of the top surface 301. As will be discussed below, in one embodiment, the lip 303 engages with a groove on the inside of the outer frame. In one embodiment, the lip 303 has a height 315A of approximately 6.6 mm along the direction perpendicular to the top surface 301 of the insert. In one embodiment, the total height 313A of the insert 300 may be approximately 60.3 mm. In one embodiment, the total width 313B of the top surface 301 of the insert 300 may be approximately 92.3 mm In one embodiment, the lip may have a width 315B of approximately 3.3 mm such that it extends approximately 3.3 mm outwards from the perimeter of the top surface 301. In one embodiment, the insert 300 may have a length 313C of approximately 220.9 mm. Although example dimensions are provided, it is contemplated that any dimensions suitable to construct an insert that holds a medical device may be used.

Each of the one or more support members 305 may have a first end located at the base of a cavity 307 or the top surface 301 of the insert 300. The second ends 311 of the one or more support members 305 may be located a distance away from the top surface and form a base for the insert. The support members 305 may provide structure and rigidity to the insert 300. The support members 305 may assist in holding the medical device in place and preventing the clean medical device case from being dislodged. Although support members 305 illustrated in FIGS. 7A-7E are pillar-like, it is envisioned that any structure suitable for providing support to the top surface 301 of the insert 300 may be used.

As illustrated in FIG. 7E, which provides a bottom view of the insert depicted in FIGS. 7A-7D, in one embodiment, the support members 305 may be distributed to form a generally x-shaped configuration. The x-shaped configuration may be sufficient to support a medical device held by the top surface 301 of the insert 300. The x-shaped configuration may also be sufficient to stabilize the clean medical device case having the embedded insert 300 by adding weight that prevents the clean medical device case from being dislodged. At the same time the weight added by the x-shaped configuration does increase the weight of the clean medical device case such that it cannot be easily moved by hospital personnel. Furthermore, the x-shaped configuration of the support members 305 may reduce the amount of material required to construct the insert 300, thereby reducing the cost of the manufacturing the insert 300. Alternatively, in one embodiment the support member 305 may be a single solid formation that forms a generally rectangular shape (such as a block) under the top surface 301 of the insert 300. In such an embodiment, the clean medical device case having the embedded insert 300 will increase the total weight of the materials used to construct the insert 300. However, depending on the material used, the weight increase may not be substantial. Accordingly, the support members may be constructed so as to balance (a) the strength necessary for the medical device case to protect its contents, with (b) the weight necessary to allow personnel to transport the medical device case, as well as (c) the cost of materials used to manufacture the medical device case.

In one embodiment, the components of the insert 300 comprise separate pieces that are fused together to form a single insert. Alternatively, the components of the insert 300 may be manufactured as a single piece through injection molding or a similar process.

The insert 300 may be constructed from silicone, plastic, or any other materials suitable for sterilization in accordance with the protocols discussed below. In one embodiment, the insert may be constructed by first constructing a negative mold of the medical device. In a second step, an injection molding system that utilizes the negative mold may construct the insert 300 such that the top surface 301 of the insert conforms to the shape of the medical device as depicted by the negative mold. Alternatively, the one or more components of the insert 300 may be 3D printed. In one embodiment, the insert 300 is constructed from a two component silicone elastomer such as BLUESIL™ RTV 3040A (manufactured by BlueStar Silicones, York, S.C.) and the insert 300 is injection molded by SilcoTech (York, S.C.).

In one embodiment, the silicone insert has a weight of approximately 1000 grams.

As illustrated at least in FIGS. 1 and 5, in one embodiment, the insert described with respect to FIGS. 7A-7E may be embedded within the outer frame described with respect to FIGS. 1-6. In one embodiment, a clean medical device case 100 is manufactured by creating an insert 103, 300 (described above) configured to hold a medical device 111, engaging and adhering the insert 103, 300 to a first groove 117 of an outer frame 101 comprising one or more panels 135, 127A, 127B, 129, 131, and a first portion of a locking mechanism 125A. The outer frame 101 is constructed about the insert 103, 300 such that the insert 103, 300 is embedded within the outer frame 101. Additionally, a lid 105 having a second portion of the locking mechanism 125B is constructed such that the lid 105 may engage with the second groove 121 of the outer frame 101. The lid 105 may be held in place with respect to the outer frame 101 when the first portion of the locking mechanism 125A is engaged with the second portion of the locking mechanism 125B.

In one embodiment, the grooves on each of the lateral panels that make up the first groove 117 on the inner surface 119 of the outer frame 101 may each have an opening that faces the interior of the outer frame 101 that is smaller than the height of the lip 303 of the insert 103, 300. In such an embodiment, the insert 103, 300 may be semi-flexible such that it can be squeezed into the first groove 117. Once placed within the first groove 117, the lip 303 of the insert 103, 300 may then expand, such that the insert 103, 300 cannot be removed from the outer frame 101. In one embodiment, an adhesive may be applied to the lip 303 of the insert 103, 300 prior to its insertion into the first groove 117 of the outer frame 101. Alternatively, or additionally, adhesive may be applied to the first groove 117 of the outer frame 101. For example, a silicone adhesive capable of affixing HDPE to silicone may be used between the lip 303 of the insert 103, 300 and the first groove 117 of the outer frame 101.

The outer frame 101 may be manufactured around the insert 103, 300. In one embodiment each of the separate lateral panels (described above, see two long side panels 127A, 127B, rear short side panel 129, and front short side panel 131) may be aligned with corresponding portions of the insert 103, 300. Optionally, an adhesive may be applied to the space between the panels and the insert such as, for example, a suitable glue, epoxy, silicone adhesive, or the like. For example, a silicone adhesive capable of affixing HDPE to silicone may be used between the panels and the insert. Similarly, a silicone adhesive of affixing HDPE to HDPE may be applied between adjacent panels.

In one embodiment, the adhesive may form an additional sealant layer between the insert and outer frame thereby contributing to maintaining a sterile area for the medical device. The same adhesive or a second adhesive may be applied to the space between each of the panels. Suitable adhesives for bonding the outer frame to the insert may include solvent-free, acrylic-based, structural adhesives. One or more clamps may be used to hold the panels in place with respect to the insert and/or the other panels while the adhesive is applied and/or the panels are welded together.

Using a welding process, the adhesive may be heated such that the panels that form the outer frame are fixed with respect to its adjacent panels. As the outer frame is constructed around the insert, there is minimal to no space between the insert and the outer frame. The outer frame may be constructed around the insert in a two-step process. In a first step, the lateral panels of the outer frame (the two long side panels 127A, 127B, the rear short side panel 129, and the front short side panel 131) may be welded to each other while being clamped to each other and/or to the insert. In a second step, the fifth panel 135 may be welded to the lateral panels of the outer frame. Accordingly, there is minimal to no space for non-sterile particulates including bacteria, viruses, protozoa, and the like to reside.

The welding process helps keep the area that houses the medical device 111 sterile from outside contaminants by eliminating, or substantially eliminating, voids in-between the panels. However, other suitable joining means may be used to assemble clean medical device case 100 as long as the internal cavity can remain sterile from outside contaminants entering through voids created by the joining process. For example, in one embodiment, flame etching may be used by igniting propane gas and using the flame to heat the plastic to align molecules on the plastic panels before attaching them together with adhesive.

In one embodiment, the welding process may include a plastic injection welding process. A plastic injection welding process may include the steps of placing a softened piece of plastic material such as polypropylene in the cavity formed between the two or more panels. The area may be heated to form a cohesive bond between the panel, poplypropylene and second piece (i.e., panel or insert). Alternative welding methods including extrusion welding and hot gas welding, may be used. Some of the gases used in the welding process may include, for example, shielding gases such as carbon dioxide, argon, helium, fuel gases such as acetylene, propane, butane, and oxygen mixtures.

In one embodiment, caulking or a sealant may be applied about the perimeter formed by the top surface of the insert 103, 300 near the first groove 117 of the outer frame 101 in order to provide additional sealing and protection of the lower sterile compartment.

While an insert that is non-removably embedded within the outer frame is described, it is contemplated that in some embodiments, the insert may be removable from the outer frame.

The clean medical device case illustrated in FIGS. 1-7E may be utilized in a hospital operating room or similar environment. A medical device may be contained within the depicted clean medical device case. When the medical device is needed, the lid 105 may be removed or disengaged from the outer frame 101, thus exposing the medical device. Hospital personnel may use the medical device while the clean medical device case 100 remains on the operating table or other surface. The friction feet depicted in FIG. 6 may be used to stabilize the clean medical device case 100 on the operating table or other surface. After use, the clean medical device 111, which is no longer sterile, may be returned to the clean medical device case 100 for cleaning. The case 100 and the medical device 111 may then be cleaned in accordance with the protocols discussed below.

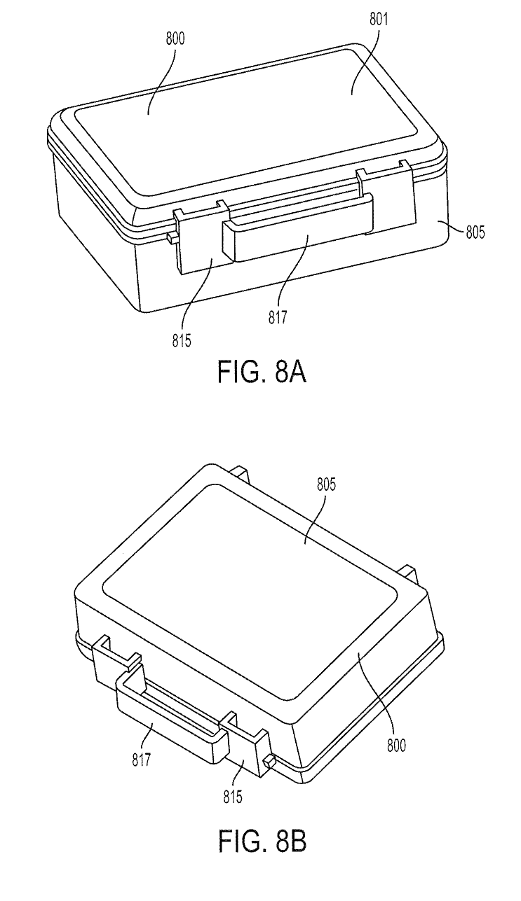

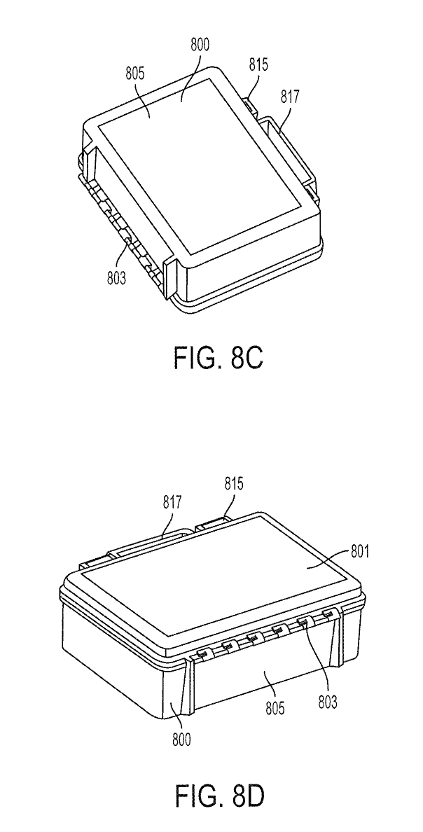

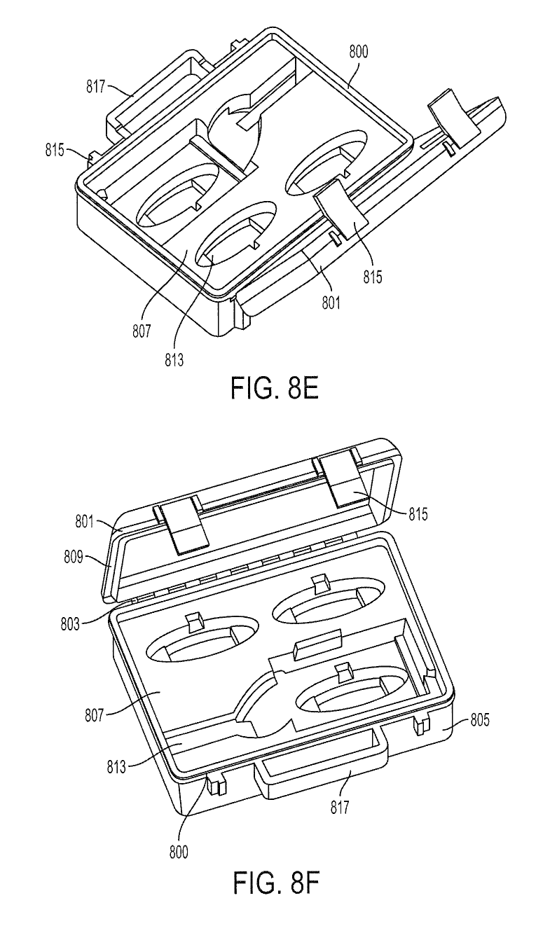

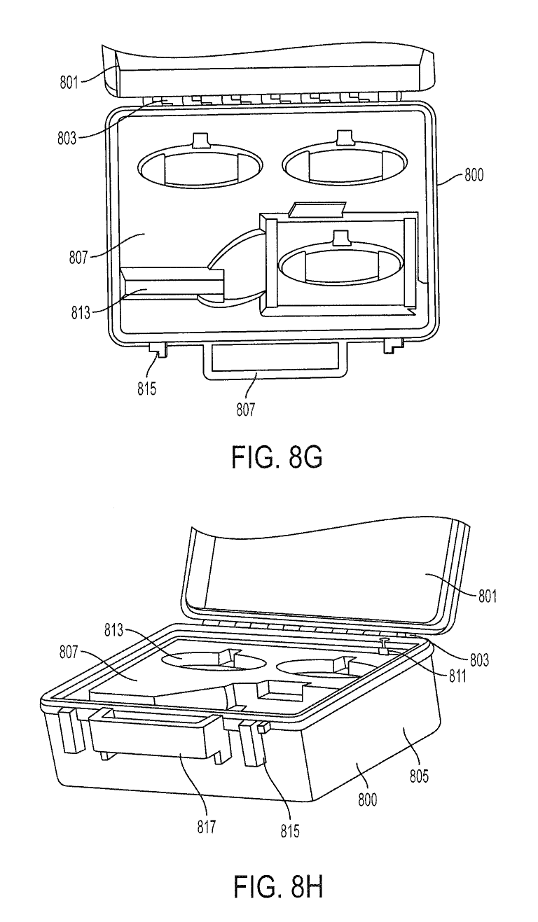

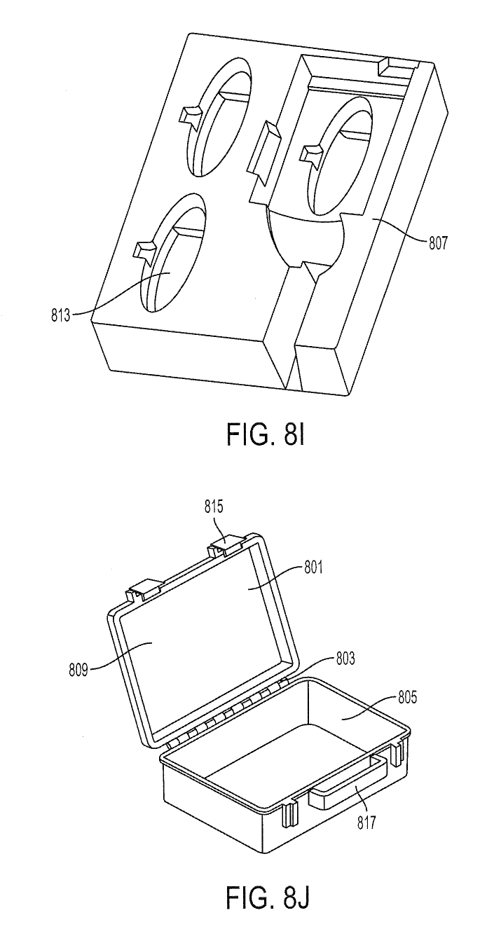

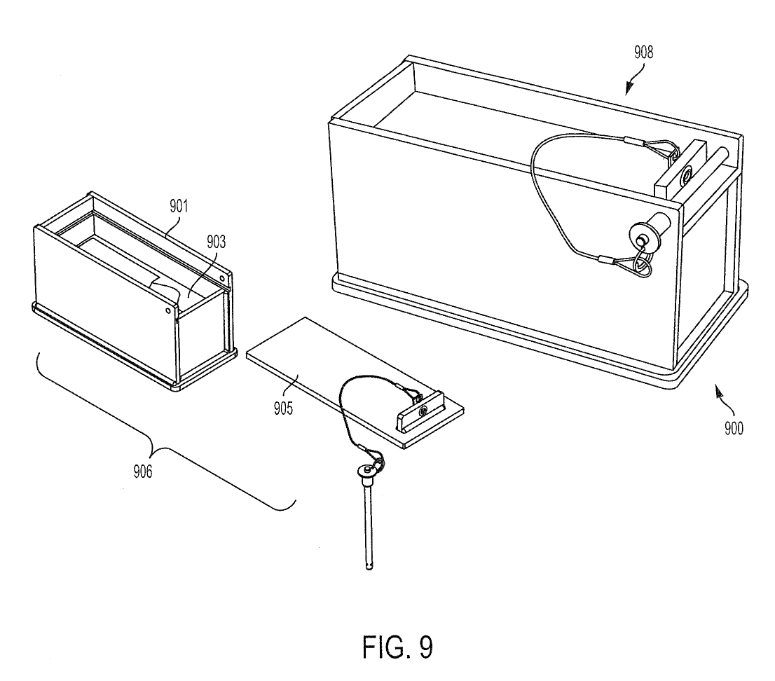

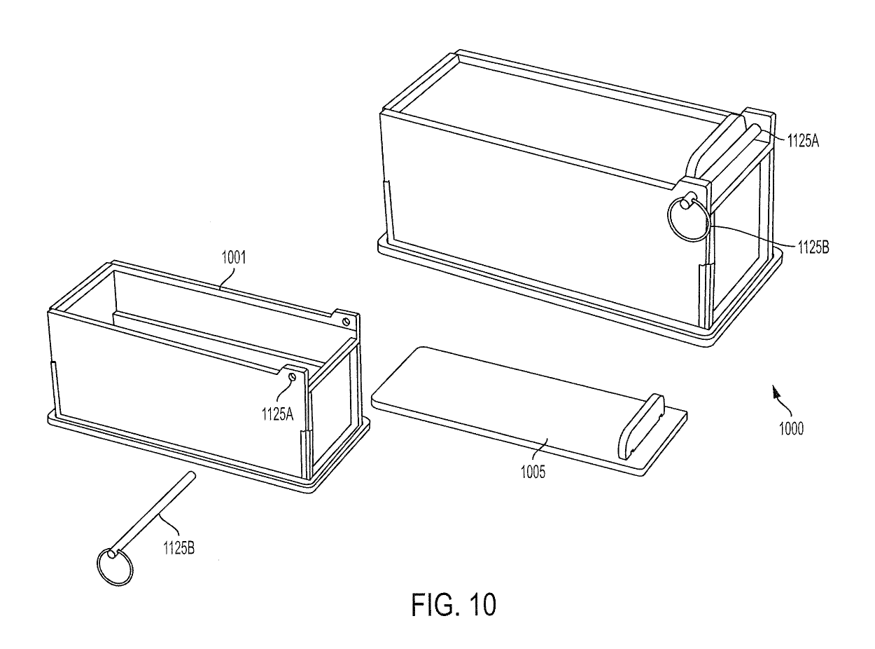

An ambulatory embodiment of a clean medical device case is illustrated in FIGS. 8A-8J. As depicted, clean medical device case 800 includes an upper section 801 hingedly 803 connected to a lower section 805. The upper 801 and lower 805 sections may form a seal when the upper 801 and lower 805 sections pivot towards each other and become engaged/locked, activating an internal silicone seal between the upper 801 and lower 805 sections of the case 800. In one embodiment, the internal silicone seal may be embedded in at least one of the case sections 801, 805. When the upper 801 and lower 805 sections of the case 800 pivot towards each other, they become engaged, and then locked using lock(s) 815. The internal silicone seal may provide an internal air and water tight seal that in one embodiment, and is air and water tight to a depth of approximately 33 feet. An insert 807 may be embedded within the lower section 805. The insert 807 may be configured to hold a medical device. The inner surface 809 of the upper section 801, the insert 807, and an upper portion 811 of the lower section 805 may form a sterile environment. The clean medical device case 800 may include a lock 815 to hold the upper section 801 in place with the lower section 805 such that the sterile environment is not exposed. The clean medical device case 800 may include a handle 817 so that the clean medical device case 800 may be easily transported.

The insert 807 may be constructed in accordance with the methods and materials described above. For example, the insert 807 may include one or more cavities 813 configured to conform to the shape of a medical device. The insert 807 may contain additional cavities configured to hold items that are beneficial for an ambulatory device such as extra parts and batteries for the medical device. The insert 807 may be manufactured from silicone by injection molding. Alternative materials and processes may be used.

The insert 807 may include two layers. A first layer composed of form-fitting plastic (such as HDPE) may be covered by a second layer composed of silicone. The silicone may have a thickness between about 5-6 mm. Alternative thickness may also be used. The first layer and the second layer may be joined together by adhesives or other means to ensure sterility between the two component parts.

Although a hinged connection is illustrated in FIGS. 8A-8J, alternative configurations may be used.

Similar to the process described above with respect to a first embodiment of the clean medical device case, the upper 801 and lower 805 sections of the clean medical device case 800 may be manufactured from HDPE in multiple panels. The panels may be shaped to conform to the surfaces formed by the insert 807. The panels may be clamped, and welded to the shape of the insert 807. The clean medical device case described in the embodiments above may be cleaned using a wiping method and/or a soaking method.

In a wiping method, first the medical device and any related accessories may be removed from the clean medical device case. Then, a cloth may be dampened with a cleaning agent. The cleaning agent may include, for example, mild soap and water, 70% isopropyl alcohol, 3% hydrogen peroxide, 10% chlorine bleach (sodium hypochlorite) prepared within 24 hours, Lysol®, Windex®, Cidex®, CaviCide®, and the like. Then the dampened cloth may be used to wipe all exposed surfaces of the clean medical device case, including the insert and cavities. The cleaned surfaces may then be wiped with distilled water in order to remove any cleaning solution and/or residue. Alternatively, PDI Super Sani-Cloth Wipes® and the like may be used. The clean medical device may then be air-dried or hand-dried using a clean, absorbent, non-shedding lint-free cloth. A clean medical device and its accessories may be placed inside the outer frame of the medical device, the lid may be engaged with the outer frame and then locked. The assembly may be transported and then stored in the proper location.

In a soaking method, first the medical device and any related accessories may be removed from the clean medical device case. A first receptacle may be filled with a cleaning agent. The cleaning agent may include, for example, mild soap and water, 70% isopropyl alcohol, 3% hydrogen peroxide, 10% chlorine bleach (sodium hypochlorite) prepared within 24 hours, Lysol®, Windex®, Cidex®, and CaviCide®. The clean medical device case may be immersed within the first receptacle. The clean medical device case may be immersed in the first receptacle such that any air trapped within the medical device case is dislodged. Then the clean medical device case may be agitated within the solution. A lid may be placed on the receptacle and the medical device case may be soaked for some period of time (e.g., five minutes). After the period of time has elapsed, the medical device case may be removed from the first receptacle and drained before being transported to a second receptacle.

In the second receptacle, the clean medical device case may be rinsed with clean or distilled water. In one embodiment, the clean medical device case may be immersed within the second receptacle. After the medical device case is removed, it may be air-dried or hand-dried using a clean, absorbent, non-shedding lint-free cloth. A clean medical device and its accessories may be placed inside the outer frame of the medical device, the lid may be engaged with the outer frame and then locked. The assembly may be transported and then stored in the proper location. The soaking method may be the preferred method of cleaning the clean medical device case.

Various illustrations of the embodiments of the clean medical device case discussed above are included in FIGS. 9-19. FIG. 9 illustrates an embodiment of the clean medical device case 900. In the illustrated embodiment an outer frame 901 with insert 903 and lid 905 in an unengaged state 906 and an engaged state 908.

FIG. 10 illustrates another embodiment of the a clean medical device case 1000. In the illustrated embodiment, an outer frame 1001 is shown without an insert. The lid 1005 may engage with the outer frame 1001 as discussed above. A locking mechanism 1125B separate from the lid 1005 may engage with a locking component 1125A on the outer frame 1001 to reversibly couple the lid 1005 to the outer frame 1001.

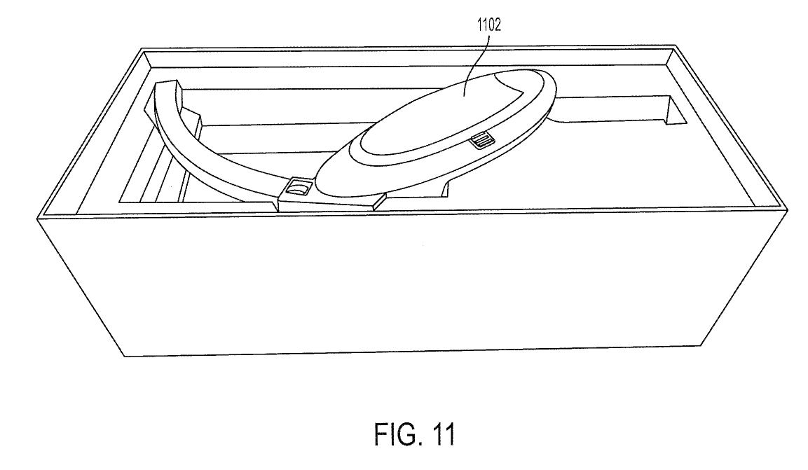

FIG. 11 illustrates a medical device 1102 as provided in its original non-sterile packaging.

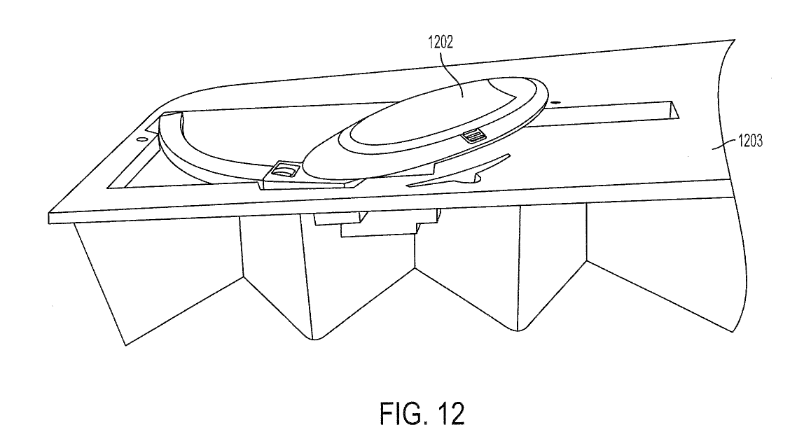

FIG. 12 illustrates a medical device 1202 placed within an insert 1203 of a clean medical device case.

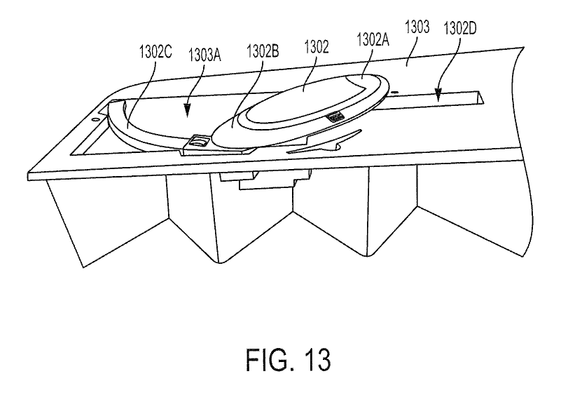

FIG. 13 illustrates a medical device 1302 placed within an insert 1303 of a clean medical device case. As discussed above, the insert 1303 medical device 1302 may be sized and configured to hold one or more components of the medical device 1302. For example, the insert 1303 may include an area 1303A configured to receive a spare battery. Components of the medical device 1302 include an attached battery 1302A, a computer 1302B, a slim blade and/or a variety of sanitary disposable blades and/or extensions that attach to the slim blade 1302C, and a TV monitor and/or integrated vertical screen 1302D. As illustrated, the components of the medical device 1302 listed above may be stored within the insert 1303 of the clean medical device case.

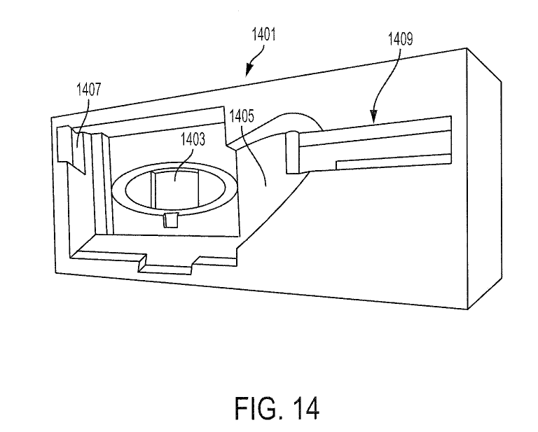

FIG. 14 illustrates an insert 1401 of a clean medical device case configured to receive a medical device. The insert 1401 may have one or more cavities configured to receive the medical device. For example, a cavity 1407 may be configured to store a slim blade of the medical device. A cavity 1405 may be configured to store a computer of the medical device. A cavity 1403 may be configured to store a spare battery of the medical device. And a cavity 1409 may be configured to store a TV monitor, or integrated vertical screen of the medical device.

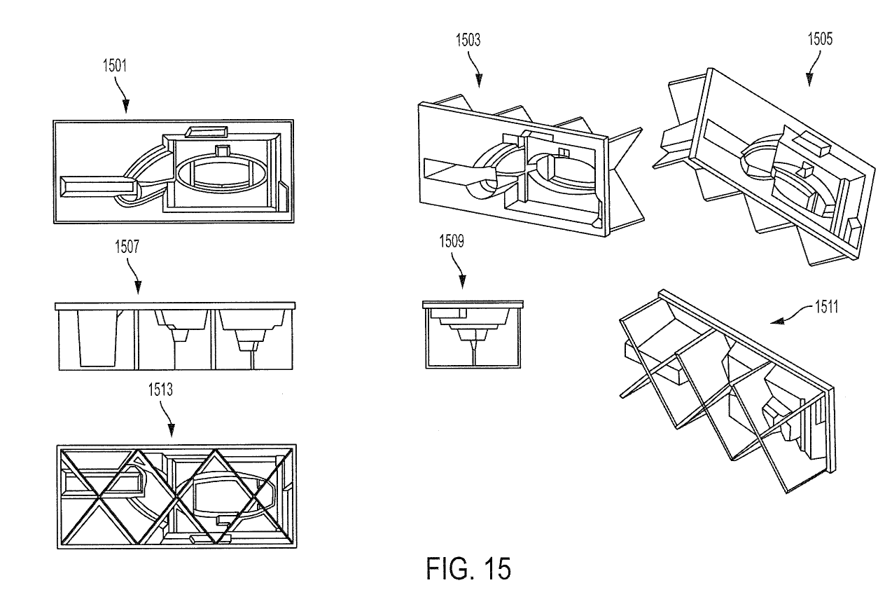

FIG. 15 illustrates various views for an insert of a clean medical device case. View 1501 illustrates a top view of the insert. View 1503 illustrates a top perspective view of the insert. View 1505 illustrates a second top perspective view of the insert. View 1507 illustrates a side view of the insert. View 1509 illustrates a second side view of the insert. View 1511 illustrates a bottom perspective view of the insert. View 1513 illustrates a bottom view of the insert.

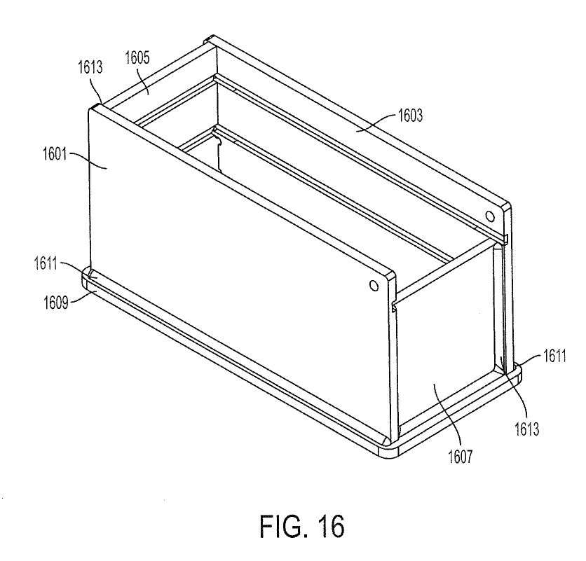

FIG. 16 illustrates specifications for an outer frame of a clean medical device case. As illustrated, the outer frame may include a side right panel 1601, a side left panel 1603, a back panel 1605, a front panel 1607, a bottom panel 1609, a welded side panel 1611, and a welded end panel 1613. In one embodiment, the side right panel 1601 may weigh approximately about 152.5 grams, the side left panel 1603 may weigh approximately about 152.6 grams, a back panel 1605 may weigh approximately 57 grams, a front panel 1607 may weigh approximately about 45.5 grams, a bottom panel 1609 may weigh approximately 163.1 grams, a welded side panel 1611 may weigh approximately 2.1 grams, and a welded end panel 1613 may weigh approximately 2.3 grams. In one embodiment, weights may vary in the range of 5% to 15% off the provided specifications.

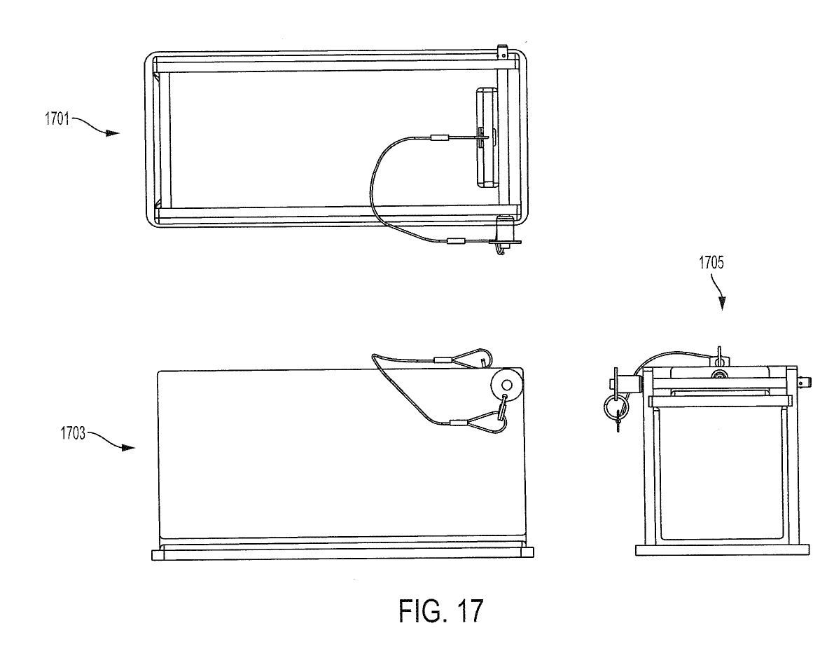

FIG. 17 illustrates specifications for a lid and outer frame of a clean medical device case. A top view is illustrated in view 1701. A first side view is illustrated in view 1703. A second side view is illustrated in view 1705.

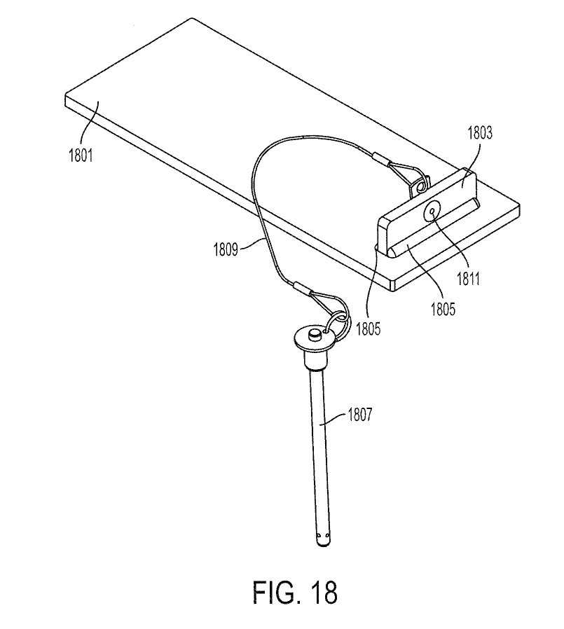

FIG. 18 illustrates specifications for a lid of a clean medical device case. As illustrated, the lid 1801 may include a pull 1803, a welded portion 1805, a pin component 1807, a lanyard component 1809, and a rivet 1811. In the illustrated embodiment, the lid 1801 may weight approximately about 124.73 grams, the pull 1803 may weigh approximately about 0.02 grams, and the welded portion 1805 may weigh approximately about 0.6 grams. In one embodiment weights may vary in the range of 5% to 15% off the provided specifications.

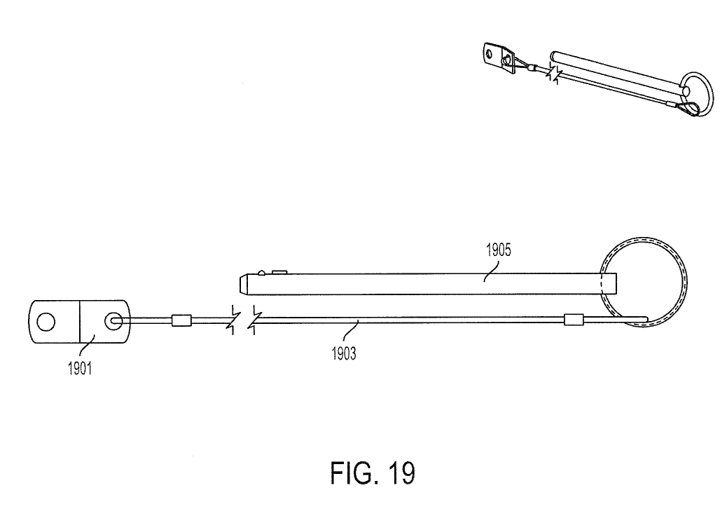

FIG. 19 illustrates specifications for a locking mechanism used in connection with a clean medical device case. As illustrated the locking mechanism may include an attachment portion 1901, a cord (also referred to as a lanyard) 1903, and a pin 1905.

While the present disclosure has been discussed in term of certain embodiments, it should be appreciated that the present disclosure is not so limited. The embodiments are explained herein by way of example, and there are numerous modifications, variations and other embodiments that may be employed that would still be within the scope of the present disclosure.

I claim:

1. A clean medical device case comprising:
an insert comprising
a lip about the perimeter of the insert, and
a body region configured to hold a medical device;
an outer frame having a first end spaced apart from a second end, the outer frame further comprising
a first groove along an inner surface of the outer frame dividing the outer frame into an upper portion and a lower portion, the first groove configured to engage with the lip of the insert;
a second groove along the inner surface of the outer frame proximate the first end of the outer frame; and
a lid configured to releasably attach to the outer frame and engage with the second groove,
wherein the inner surface of the upper portion of the outer frame, the body region of the insert, and an interior surface of the lid faun a sterile environment.

2. A clean medical device case comprising:
an upper section hingedly connected to a lower section, the upper and lower sections forming a seal when the upper and lower sections are pivoted towards each together;
the lower section having an insert embedded within the lower section, the insert configured to hold a medical device;
wherein the inner surface of the upper section, the insert, and an upper portion of the lower section extending between the insert and an interface between the upper section and the lower section are configured to form a sterile environment.

3. A clean medical device case comprising:
an insert comprising
a lip about the perimeter of the insert, and
a body region configured to hold a medical device having one or more accessories;
an outer frame having a first end spaced apart from a second end, the outer frame further comprising
a first groove along an inner surface of the outer frame dividing the outer frame into an upper portion and a lower portion, the first groove configured to engage with the lip of the insert;
a second groove along the inner surface of the outer frame proximate the first end of the outer frame; and
a lid configured to releasably attach to the outer frame and engage with the second groove,
wherein the inner surface of the upper portion of the outer frame, the body region of the insert, and an interior surface of the lid form a first sterile environment, and
the inner surface of the lower portion of the outer frame, a bottom surface of the insert and a bottom surface of the outer frame form a second sterile environment.

4. A clean medical device case comprising:
an upper section hingedly connected to a lower section, the upper and lower sections forming a seal when the upper and lower sections are pivoted towards each together;
the lower section having an insert embedded within the lower section, the insert configured to hold a medical device having one or more accessories;
wherein the inner surface of the upper section, the insert, and an upper portion of the lower section extending between the insert and an interface between the upper section and the lower section are configured to form a sterile environment, and
the inner surface of the lower portion of the outer frame, a bottom surface of the insert and a bottom surface of the outer frame form a second sterile environment.
